(19) United States
(12) Patent Application Publication
(10) Pub. No.: US 2018/0317783 A1
(43) Pub. Date: Nov. 8, 2018

(54) FETAL HEART MONITOR VESTMENT

(71) Applicants: BORIS PETRIKOVSKY, GREAT NECK, NY (US); JUAN M. VILLAR, HANOVER, NH (US)

(72) Inventors: BORIS PETRIKOVSKY, GREAT NECK, NY (US); JUAN M. VILLAR, HANOVER, NH (US)

(21) Appl. No.: 15/784,123

(22) Filed: Oct. 14, 2017

Related U.S. Application Data
(60) Provisional application No. 62/500,184, filed on May 2, 2017.

(51) Int. Cl. A61B 5/024; A61B 5/0444; A61B 5/00

(52) U.S. Cl. CPC A61B 5/02411; A61B 5/0444; A61B 5/6804; A61B 5/02438; A61B 5/4362; A61B 5/0011; A61B 5/7257

(57) ABSTRACT

Disclosed is a fetal heart monitor vestment, having a non-conductive fabric garment configured to be closely fitted to a female, a monitor controller unit comprising a fetal heart rate monitor and having a data input port, an ECG harness comprising a plurality of electrodes and a plurality of conductive fabric wires, each electrode attached to at least one of the conductive fabric wires, the conductive fabric wires in attachment to and in communication with the data input port, the electrodes and conductive fabric wires integrated into the fabric of the garment, and at least one said electrode disposed in the garment so as to be positioned on, and in close contact with, the female in a manner effective in sensing a fetal cardiac potential signal. A variety of fetal heart rate monitors and methods of operation are also disclosed.

### CROSS-REFERENCE TO RELATED APPLICATION

This application claims the benefit of U.S. Provisional Application No. 62/500,184, filed May 2, 2017, Confirmation No. 8211, the disclosures of which are incorporated by reference herein in their entirety.

### BACKGROUND OF THE DISCLOSURE

### Field of the Disclosure

This invention relates to the field of garments having sensors integrated therein for detecting and monitoring the heartbeat of a fetus during pregnancy.

### BRIEF DESCRIPTION OF THE DISCLOSURE

Disclosed is a fetal heart monitor vestment, having a non-conductive fabric garment configured to be closely fitted to a female, a monitor controller unit comprising a fetal heart rate monitor and having a data input port, an ECG harness comprising a plurality of electrodes and a plurality of conductive fabric wires, each electrode attached to at least one of the conductive fabric wires, the conductive fabric wires in attachment to and in communication with the data input port, the electrodes and conductive fabric wires integrated into the fabric of the garment, and at least one said electrode disposed in the garment so as to be positioned on, and in close contact with, the female in a manner effective in sensing a fetal cardiac potential signal. A variety of fetal heart rate monitors and methods of operation are also disclosed.

Also disclosed is a kit having a plurality of garments of increasing size and at least one removable monitor controller unit.

### BRIEF DESCRIPTION OF THE DRAWINGS

FIG. 1 Is a front elevation view of an embodiment of a garment of the disclosure.

FIG. 2 is a back elevation view of the garment of FIG. 1.

FIG. 3 is a close-up view of a monitor controller and abdominal electrode connections.

FIG. 4 is a cross-sectional view of an electrode and fabric wire electrically stitched together through the garment fabric.

FIG. 4b is a close-up cross-sectional view of a lock stitching.

FIG. 5 shows examples of abdominal, maternal, and fetal ECG signals and includes an INSET diagram of the PQRST wave labelling convention for ECG signals.

FIG. 6 is a schematic of an embodiment of the disclosure utilizing direct filtering without adaptive noise cancellation.

FIG. 7 is a schematic of an embodiment of the disclosure utilizing adaptive filtering with noise cancellation.

FIG. 8 is a schematic of an embodiment of the disclosure utilizing Fast Fourier Transform (FFT) adaptive filtering.

FIG. 9 is a graph of fetal, maternal, and 2^{nd} harmonic maternal heartbeat rates (beats/minute) plotted against gestation age measured in weeks since the last menstrual period (LMP).

FIG. 10-A is an abdominal electrocardiogram (ECG) of a pregnant woman.

FIG. 10-B is a graph of a Fast Fourier Transform of the ECG of FIG. 10-A in the frequency domain after signal filtering.

FIG. 11 is a flowchart of a method of an FFT tracking embodiment the disclosure.

FIG. 12 is a pair of front and a back perspective drawings of an expectant mother wearing a garment sized for about 21 weeks into a pregnancy.

FIG. 13 is a pair of front and back perspective drawings of an expectant mother wearing a garment sized for about 28 weeks into a pregnancy.

FIG. 14 is a pair of front and back perspective drawings of an expectant mother wearing a garment sized for about 35 weeks into a pregnancy.

### DETAILED DESCRIPTION OF THE DISCLOSURE

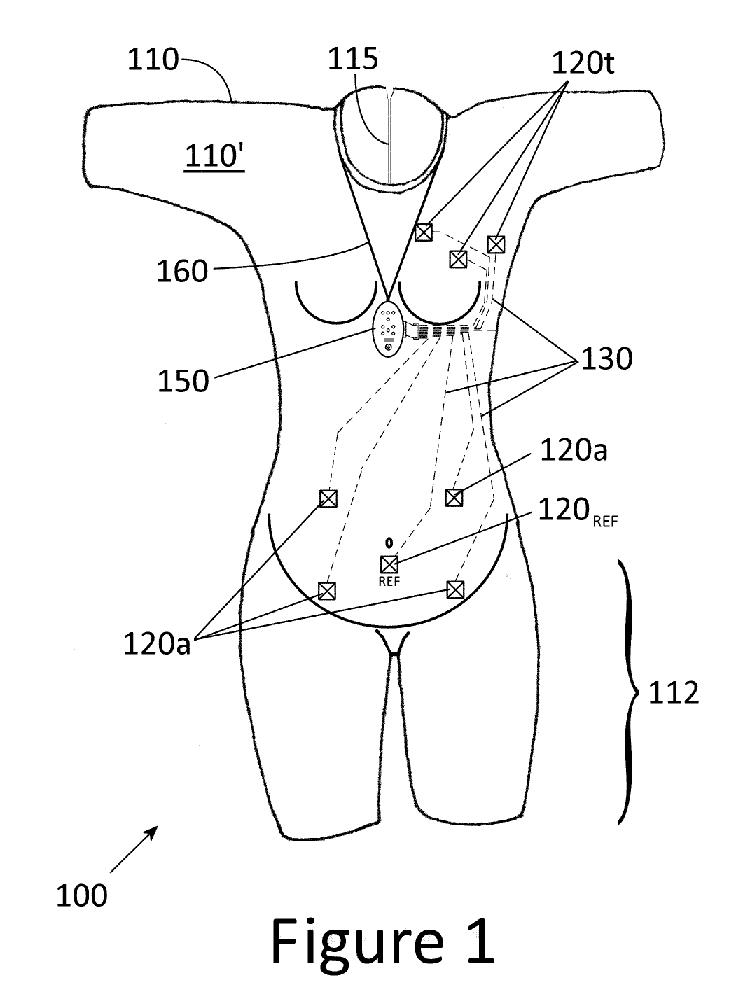

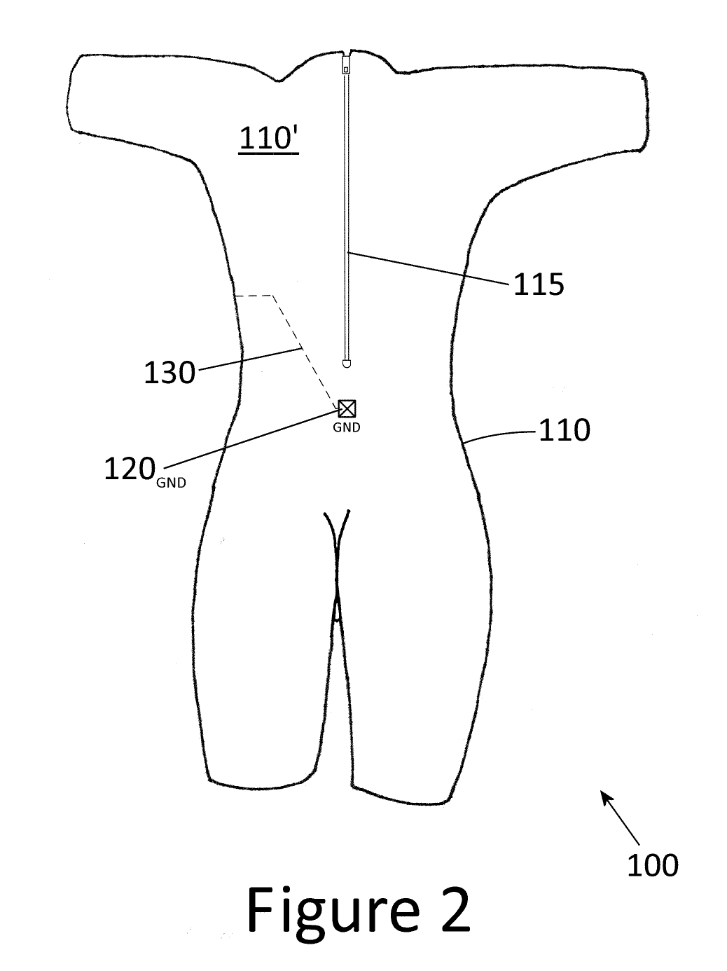

Referring to FIGS. 1 and 2, there is shown an embodiment of a fetal heart monitor vestment 100 of the disclosure in which at least one textile, or fabric 110′, is cut to pattern and assembled into a garment 110 that is configured to closely fit the contours of an expectant mother. The garment 110 is provided with a plurality of electrodes 120 integrated therewith, namely at least one abdominal electrode 120a, one or more optional thoracic electrodes 120t, a reference electrode 120_{REF}, and a ground electrode 120_{GND}.

It should be noted that the ground electrode 120_{GND} may be located anywhere on the body and the reference electrode 120_{REF}, may optionally be located near the navel.

The garment may have a pant portion 112 integrated therewith as shown or more simply be in the form of a shirt. In the former configuration, a zipper 115 or other closure may be provided.

In an embodiment to obtain close-fitting of the garment 110 to the female, the garment fabric 110′ of the garment 110 may be a stretchable fabric, for example a woven elastomer in the class of segmented copolyesters such as polyester-polyurethane copolymer fibers alone or blended with cotton or other natural fibers or synthetic polymer fibers such as polyester, nylon, acrylic, and the like.

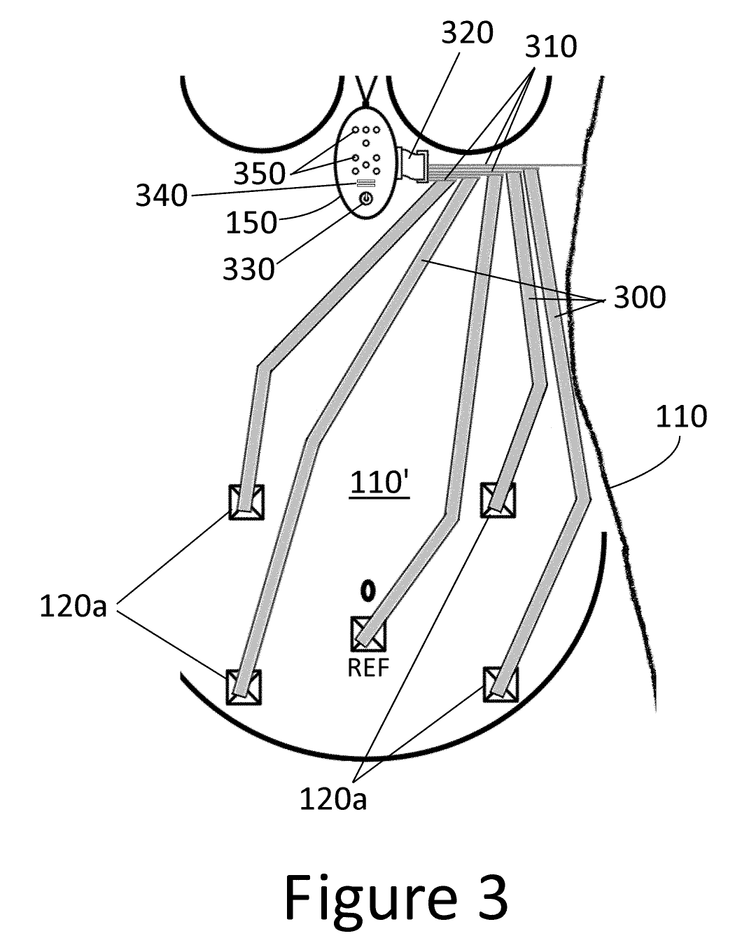

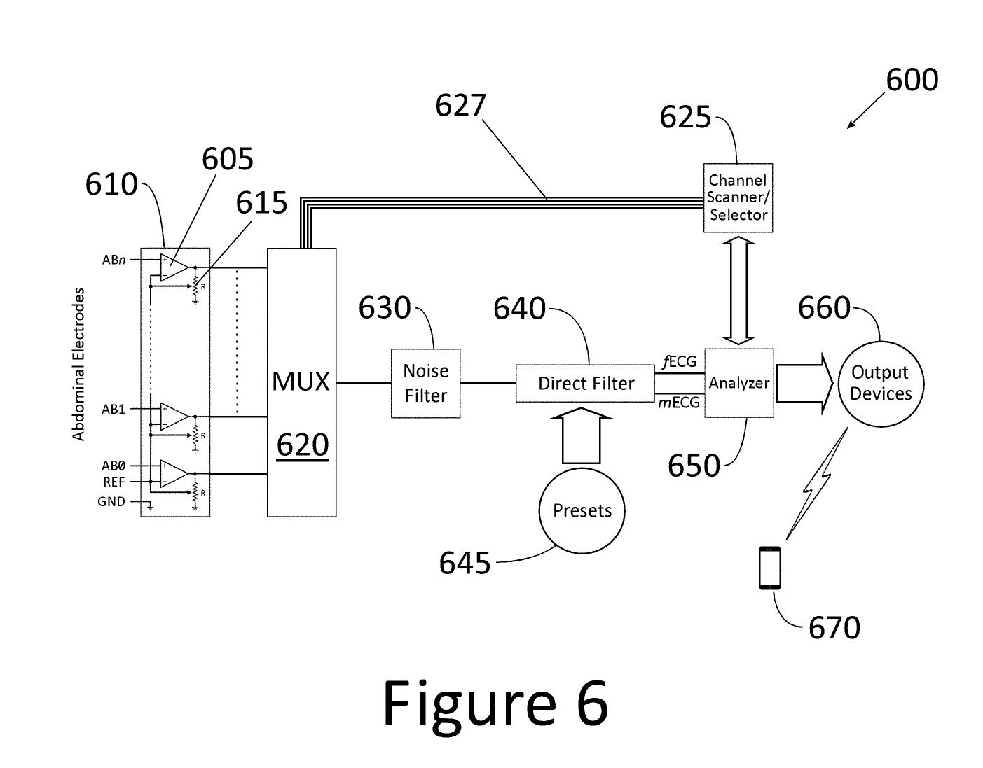

A monitor controller 150 is provided that is shown in electrical interconnection with the electrodes by way of a plurality of possible conductive pathways 130, represented by dashed lines in FIGS. 1 and 2. Within the monitor controller 150 is a fetal heart monitor 600, 700, 800 of any of the embodiments shown in FIGS. 6, 7, and 8, that receives the signals from the conductive pathways 130. These conductive pathways 130 may be embodied in the form of conductive wires sewn into the garment fabric 110′ or strips of a conductive fabric wires 300 as in FIG. 3. One or more supports 160 may be provided for the monitor controller 150, such as, for example, a hook-and-loop (e.g., Velcro) attachment directly to the garment 110 and/or a neck strap (as shown), so as to secure the monitor in place.

Referring to FIG. 3, the monitor controller 150 may be provided with a power switch 330, a speaker 340 that may be utilized to sound alerts for electrical malfunctions or cessation of the fetal or maternal heartbeat, and one or more of LED electrode status indicators 350 that light up when an electrode connection fails. In the embodiment shown, the LEDs are laid out in a pattern that echoes the pattern of the actual electrodes 120 on the garment 110 so as to make it immediately apparent to the user which electrode's status is being indicated.

Strips of conductive fabric may be utilized as fabric wires 300 sewn onto the garment 110 to electrically connect each electrode 120 to the monitor controller 150. By way of example, only connections to the reference electrode 120_{REF}, ground electrode 120_{GND}, and the abdominal electrodes 120a are shown in this configuration to avoid cluttering the drawing and demonstrate that an embodiment of this disclosure without thoracic electrodes 120t is an option as a low-cost alternative.

The fabric wires 300 will generally be made of polymer fibers that are infused or plated with a metal, usually silver, gold, copper, or stainless steel. Also known are natural or synthetic fibers interwoven with metal or carbon fiber threads. As indicated above, because of the “curviness” of the surfaces of an expectant mother, a closeness of fit may effectively be obtained by using “stretchy” material for the garment fabric 110′ and the fabric wires 300, namely an elastomer. It may be desired that the elastomeric mix of both the garment fabric 110′ and fabric wires 300 be substantially similar to be effective in preventing undue stresses upon the stitching between the two as stretch forces act upon the materials, that is to say that the garment fabric 110′ and the fabric wires 300 have the same stretch characteristics so that both stretch and deform substantially identically in response to tensile forces. If not similar in mix, then alternatively it may be desired to select elastomeric mixes that nevertheless provide similar stretch characteristics.

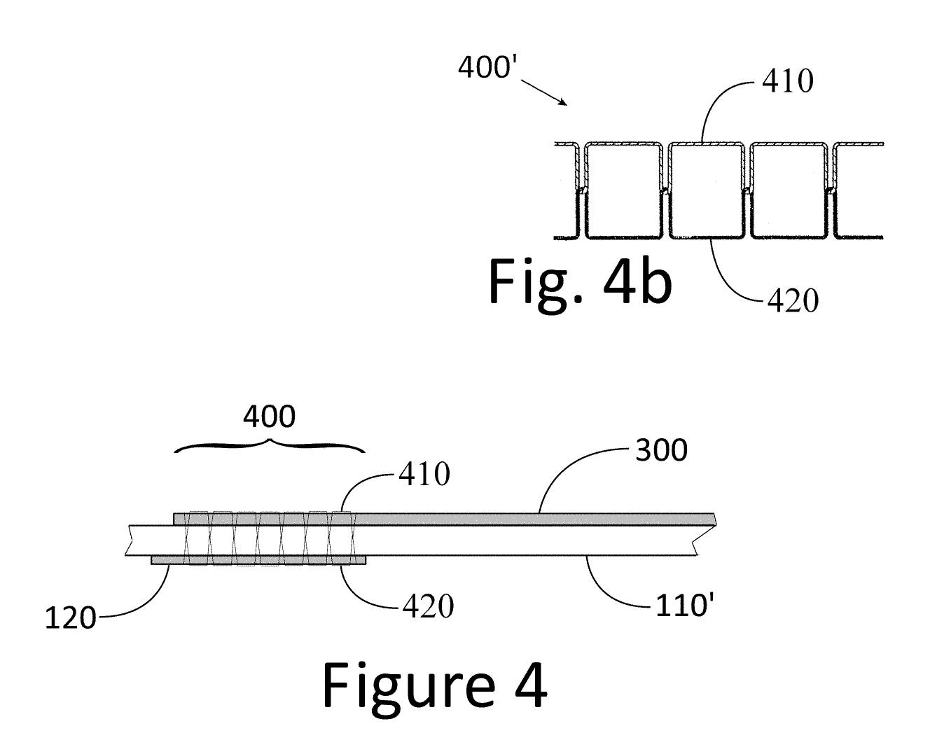

Referring to FIG. 4 there is shown a method and structure of electrically connecting a distal end of each fabric wire 300 to each electrode 120, both on opposite sides of a non-conducting segment of the garment fabric 110′. Generally, most if not all of the fabric 110′ used to make the garment 110 will be non-conductive and may be assumed to be non-conductive for the purposes of this disclosure unless indicated otherwise. One way to make the connection between the fabric wire 300 and an electrode 120 fashioned of a patch of a conductive fabric is by sewing though all three layers, fabric wire-garment-electrode, using conductive thread. A simple straight lock stitch 400′, obtainable on any home and commercial sewing machine (see FIG. 4b) having conductive thread loaded in the spool and in the bobbin will suffice, though there are any number of stitch types that will suffice. When a sewing machine stitches a material or materials, a spool thread 410 and a bobbin thread 420 interlock in one way or another depending on the type of stitch selected. If the both the spool thread 410 and the bobbin thread 420 are electrically conductive, then an electrical connection is established. As can be seen in FIG. 4, such a conductive stitching 400 not only establishes an electrical connection between the fabric wire 300 and the electrode 120, but also serves to secure (or further secure) both to the garment fabric 110′.

Alternatively, one may dispense with an electrode fashioned by a separate piece of conductive fabric and instead embroider an electrode 120 onto the underside of the garment fabric 110′ using any one or more of available embroidering stitches available on most home and commercial sewing machines.

A wide variety of electrically conductive threads are available. The most obvious “thread” is narrow gauge metal wire such as copper, silver, gold, and stainless steel wires. Stainless steel is currently popular because of its resistance to corrosion and relatively low cost. Silver coated copper wire is also available. Silver and copper threads treated with an anti-corrosion protectant are commercially available. Lower cost threads are available that are made by intertwining metal strands with polymer strands or by depositing metal upon a polymer strand, usually silver deposited upon nylon.

Note that for sewing the fabric wires 300 to the garment fabric 110′, one might desire a stretchable stitch, such as a “zig zag stitch,” or “stretch stitch,” along the length of the fabric wire 300. These types of stitches are usually found as stitch option settings on most sewing machines. One may also use an elastic thread for the purpose where needed. Another option is to glue the fabric wire 300 to the garment fabric 110′ with an appropriately stretchable adhesive, often sold as “spandex glue,” “spandex adhesive,” or “stretch fabric glue.”

Referring back to FIG. 3, the fabric wires will generally be substantially wider than simple metal wires. In order to connect to the monitor controller 150, such as by way of a bus cable 320, one may provide narrow wire connections to the monitor controller by way of bus wire stitching 310. This stitching may be accomplished by using a conductive spool thread 410 in combination with an electrically insulating bobbin thread 420 (or vice versa if it's the spool thread on the inner side of the garment at manufacture) so as to insulate the bus wire 310 from the female's skin. Note that for this purpose, just as it is for sewing on the fabric wires 300, it is not necessary that the thread stretch, only that a stretchable stitch be used, such as a zig zag stitch or a stretch stitch.

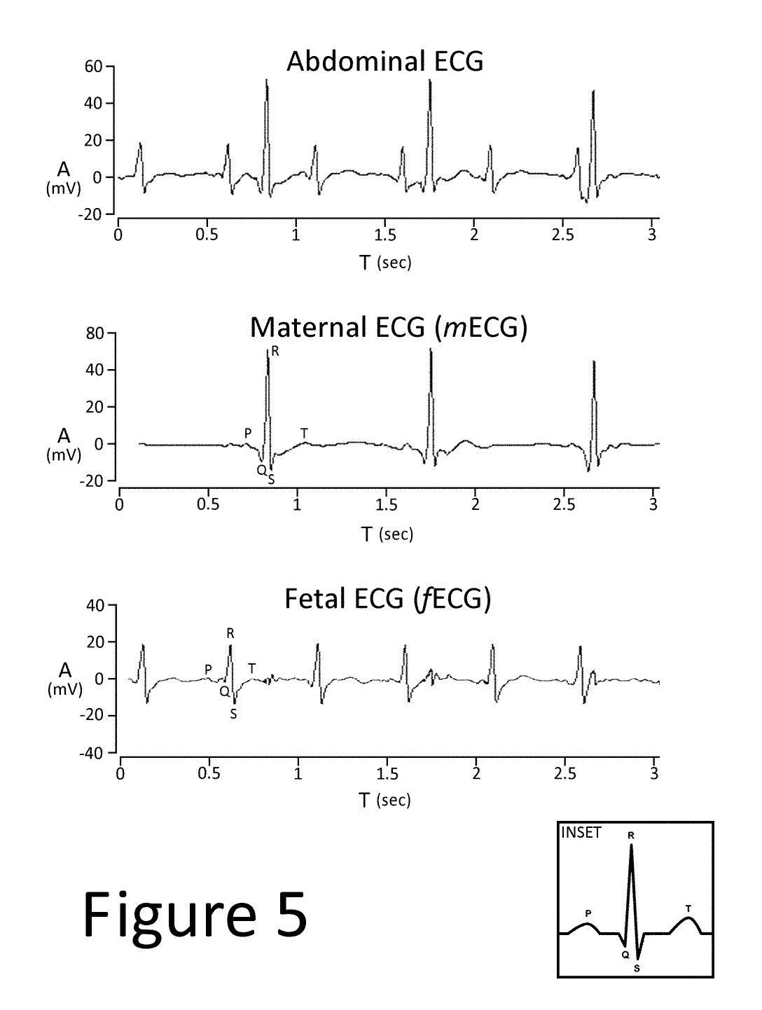

Referring to FIG. 5, there is shown three electrocardiographs (ECGs) that demonstrate the task at hand. Inputted from any of the abdominal electrodes might be something like the ECG trace labelled “Abdominal ECG” in the drawing.

The amplitude of the waveforms in the graph are very much dependent upon the position of the electrode in relation to the fetus. Note that in the final stages of pregnancy, the fetus will actually invert so as to present itself to the vaginal canal head first, which can cause what were positive amplitudes to go negative and vice-versa. Whether an amplitude in any ECG is positive or negative is dependent on the position of a lead defined by an imaginary line extending from an electrode 120 to the reference electrode 120_{REF} with relation to a depolarization vector. If a wave of depolarization of heart muscle is moving toward a lead or a wave of repolarization (negative depolarization) is moving away from a lead, then the amplitude moves positive. Reverse the directions, and the amplitude goes negative.

The objective in the analysis of abdominal ECG is to separate out the fetal ECG (fECG) from the abdominal signal. It may also be desirable to isolate the maternal ECG (mECG) to keep track of the mother's status or for the purposes of noise cancellation as will be more fully explained below. Ideally one might like to sufficiently separate out the entire PQRST complex of a fetal heartbeat wave as diagrammed in the INSET of FIG. 5, or even just the QRS complex. For most diagnostic purposes at the time of this writing, it is enough that we can extract the fetal fundamental frequency f₀, which means we seek the fetal R wave, because it is the dominant harmonic.

Not shown here is ambient noise, such as “power line interference” or “mains interference,” meaning the frequency signal generated by the power utility. In the U.S., that is 60 cycles/sec, or 60 Hz. In many other countries, it's 50 Hz. Fortunately, the typical base or fundamental frequency of the mECG is roughly around 1.0 Hz at rest and the fECG is roughly around 1.5 to 2.0 Hz depending on the stage of development. If room is made in the bandwidth up to the 10th harmonic (or 9th overtone) of the fetal fundamental frequency, then a low-pass cut-off above 20 Hz and below 50 Hz will eliminate the mains noise in most of the developed world without significant attenuation the signal of interest. Notch filters are also often used for this purpose.

Referring to FIG. 6, there is shown a direct filtering fetal heart monitor 600 for isolating the fetal ECG signal (fECG) and optionally the maternal signal (mECG) that does not utilize noise-cancellation. A “direct filter” is defined as one in which noise is stripped away, thereby revealing the desired signal relatively unchanged (see Singh, A., Adaptive Noise Cancellation, Netaji Subhas Institute of Technology, 1997, the disclosures of which are incorporated by reference herein in their entirety). Here, a direct filter 640 is utilized that may be either a fixed filter or an adaptive filter. A fixed filter is one in which the filtering response relies on information about the signal known beforehand, meaning no new information is acquired during the process of filtering, while an adaptive filter is able to adjust its impulse response with little or no prior knowledge of the signal.

In this embodiment only abdominal electrodes 120a are utilized as inputs, wherein n abdominal input leads AB, labelled “AB0” to “ABn”; a reference lead REF from the reference electrode 120_{REF}; and a ground GND connected to the ground electrode 120_{GND}; are fed into an amplifier stage 610. The amplifier may be constructed of a series of op-amps 605, the gains of which may be provided with means for adjustment 615, such as a variable impedance between the output and an input, if desired. In the embodiment shown, the abdominal input leads AB are connected each to a positive input of an op-amp and the reference lead REF is connected to the negative input of all the op-amps. The ground lead GND shares common ground with the circuit. The signals from the op-amps may then be fed into a multiplexer 620.

The multiplexer 620 selects a channel to be transmitted as a single signal-source from a selected op-amp 605 to an optional noise filter 630 in response to an address signal applied from a channel scanner/selector 625. This allows an analyzer 650 to scan through and analyze the quality of the fECG and, optionally, the mECG signals obtained to eventually identify and settle upon a channel with the best signal for a task at hand.

Two primary tasks of the analyzer 650 will be measurement of a fetal heart rate/FIR and calculation of a fetal heart rate variability fHRV, which will generally be determined from detection and measurement of the R peaks of the fECG during the second and third trimesters. Variability of these peaks refers to the variation in time elapsed between R peaks, which is supposed to change as the fetus develops. Changes in fHR that are too fast or too slow, which is to say fHRV values that are too high or too low are indicators of impending diseases such as supraventricular extrasystoles, sudden infant death syndrome, and perinatal mortality (stillbirth). (see Camm & Malik et al., Guidelines: Heart rate variability . . . , European Heart Journal, 17, 354-381, 1996; and van Laar et al., Fetal heart rate variability during pregnancy, obtained from non-invasive electrocardiogram recordings, Acta Obstet Gynecol Scand., 2014 January; 93(1):93-101, the disclosures of both of which are incorporated by reference herein in their entirety).

The noise filter 630, which may be integrated into the direct filter 640, is to strip out common noise sources, such as 50 Hz and 60 Hz power line interference, which as indicated above are usually stripped out with notch or low pass filters. Another noise problem is “baseline wander” which is contamination by a DC or very low frequency signal causing the entire ECG signals to float up or down. Beginning at about 21 weeks (gestational age, corresponding to the start of the last third of the second trimester) into a pregnancy, the mECG is lower than the fECG, though it will average 60 beats/min (bpm). The literature and experience indicate that the mECG will rarely drop below 40 bpm (≈0.7 Hz). This lowest mECG represents the lowest maternal fundamental frequency mf₀ that we might reasonably expect this far into the pregnancy, so a high-pass filter with a cutoff at about 0.1 Hz or so may be sufficient here depending on the quality of the filter. Prior to 21 weeks, it is the fetal heartbeat that is the slowest, ranging from about 0.04 Hz to 0.15 Hz. If one has reason to study in those ranges, the cut-off on a high-pass filter will have to be lowered accordingly.

The direct filter 640, as mentioned above, may be of the fixed or adaptive variety. A fixed filter requires some prior knowledge of both the signal and the noise. Fixed filters are cheap and easy to construct and usually take up little space. Often, though, they are not very effective in pulling out a clean fECG trace, but in many situations a clean fECG trace is not needed, such as when afHR or a fHRV are all that are desired.

One fixed filter method that can be used is to provide the direct filter 640 with the expectant mother's heart rate mHR as an inputted preset 645 value. If the subject is relaxed and comfortable, this will be between about 1 HZ to about 1.8 HZ. The preset value is used to set the cutoff frequencies on a high-pass filter, two notch filters, and a low-pass filter to suppress the maternal signal, mf₀, and the first three harmonics, 2mf₀, 3mf₀, and 4mf₀, of the mECG.

By eliminating the predominant maternal harmonics, the fetal signal fECG then predominates, though it remains contaminated by maternal signals, but these remaining maternal components are now small in comparison to the fetal R waves. The resulting signal in the time domain can be subjected to any number of R-R peak-to-peak or zero-to-zero measurements known in the ECG art, thereby producing the sought after JHR values. Once obtained, any variations in fHR may be analyzed to obtain fHRV. A variant of this technique may be found in Lweezy et al., Extraction of fetal heart rate and fetal heart rate variability from mother's ECG signal, World Academy of Science, engineering & Technology, 54, 2009, the disclosures of which are incorporated by reference herein in their entirety. Lweezy used Chebyshev Type II high and low band pass filters (BPFs) with some good results. A more active approach would be to apply a Sallen-Key topology for the BPFs.

An adaptive filter embodiment of the direct filter 640 can be constructed by adding a feature to enable the filter to determine the maternal heart rate mHR on its own. One way is to use the signal aECG to construct an estimate of a mixing matrix S̅, which is then converged to final form through successive iterations of a process that process known as Independent Component Analysis (ICA). Arrange aECG itself as a matrix X̅, then one may solve for Ā=fEGC by

X̅=S̅Ā (1)

With analysis and monitoring performed on a continuous basis, the analyzer may continually update and output results and warning signals to any of a number of output devices 660, such as the LED indicators 350 and speaker on the monitor controller 150 or transmitting the results to a wireless remote device 670 such as a computer or iPhone. This communication may be both ways, such that controls (e.g., software “apps”) may be provided on the wireless device to control the filtering system 600, such as by transmitting inputted presets 645, adjusting the bandwidth of the direct filter 640, or the characteristics of the analyzer, or even the gain on the op-amps 605, for example. In such case, the wireless device could be described as a “wireless remote device,” but for the purposes of this specification the term “wireless remote device” is to be understood to also encompass simple passive one-way communication configurations, which may be desirable so as to prevent the average consumer from inadvertently rendering the system inoperable.

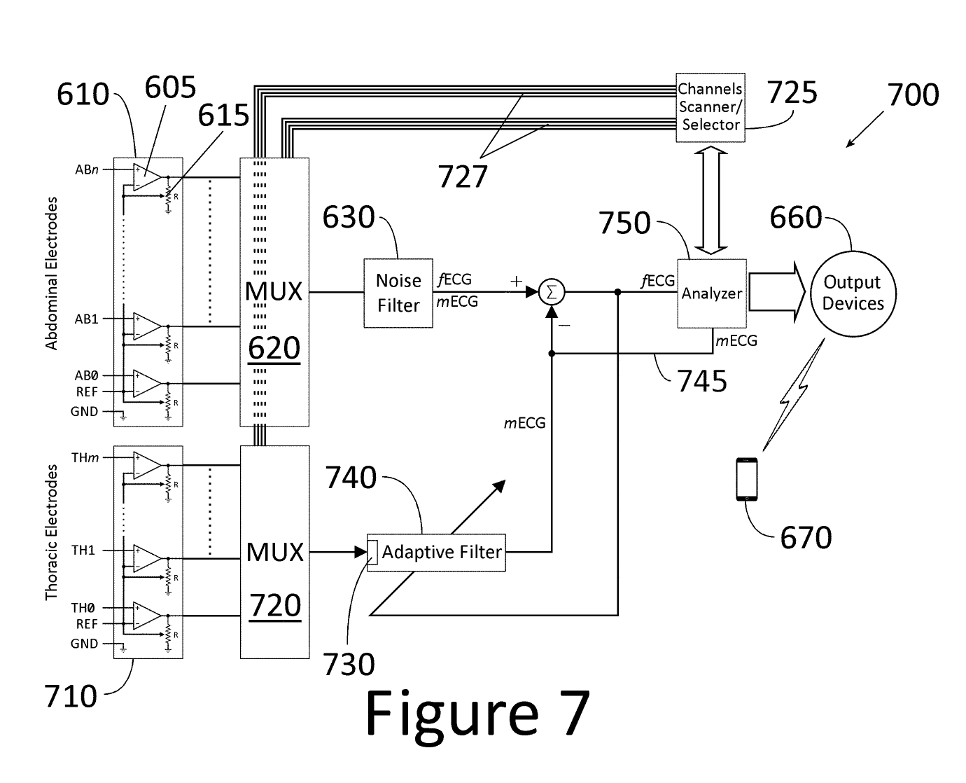

Referring to FIG. 7, an adaptive fetal heart monitor 700 embodiment is shown wherein thoracic electrodes 120t (FIG. 1) are utilized as noise-source inputs in addition to the abdominal electrodes 120a, which provide the signal-source inputs. Here, m thoracic input leads TH—labelled “TH0” to “THm”—are also fed into a thoracic amplifier stage 710. Note that while separate amplifier stages 610, 710 and multiplexers 620, 720 are shown, this is for the sake of clarity. In actual practice, the amplifier stages and multiplexer stages may be located on the same chip.

A dual channel channels scanner/selector 725 is provided to cycle through or select combinations of abdominal and thoracic inputs, under the control of the noise cancellation analyzer 750, and apply them to an adaptive filter 740. A noise filter 630 may be provided to clean up the signal to a summation node Σ, while the adaptive filter 740 has a built-in noise filter 730 to, ironically, clean noise out of the thoracic mECG signal, which itself is the noise we wish to clean out of the abdominal input signal. An additional external noise filter 630 could have just as easily been provided for the adaptive filter 740, the built in embodiment shown is by way of example.

A “copy” of the negating mECG signal may be provided to the Analyzer for further analysis or simply for reporting to one or more output devices 660, 670, by way of an mECG tap 745.

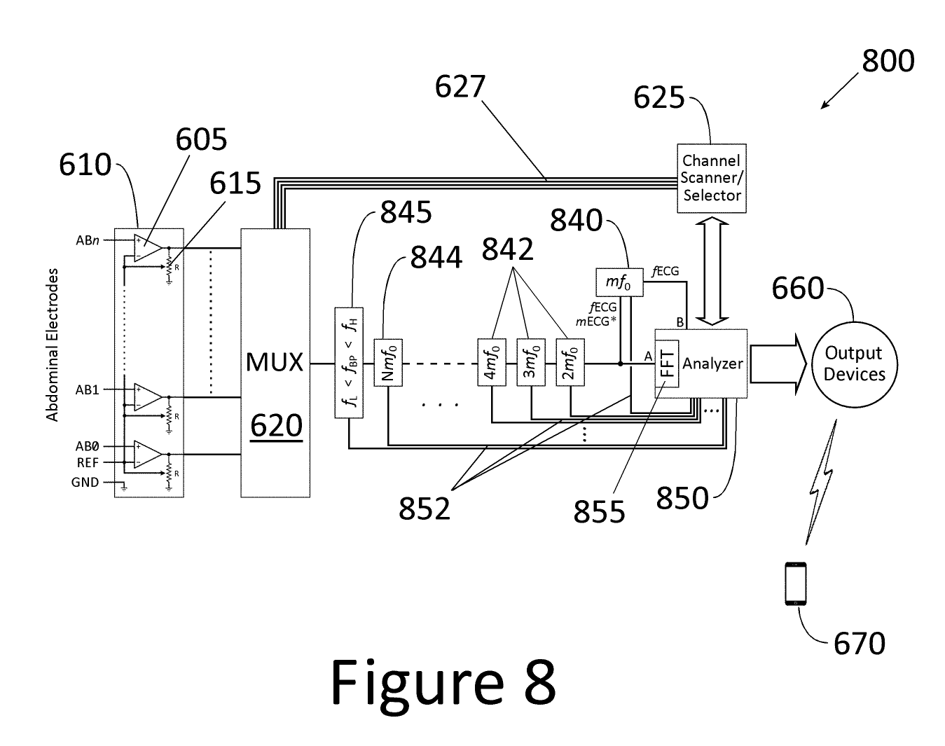

Referring to FIG. 8, a Fast Fourier Transform (FFT) fetal heart monitor 800 embodiment of the disclosure is shown. Here, an FFT-enhanced analyzer 850 is provided the services of a Fast Fourier Transform (FFT) module 855 that performs FFT on an incoming ECG signal and provides for the FFT-enhanced analyzer 850 a magnitude array (usually double floating point) containing n/2 amplitudes of Fourier components in the frequency domain that make up the signal, where n is the number of samples taken at time intervals of Δt of the ECG over a period of time known as the “window length” (=nΔt), which may typically be anywhere from 1 to 100 seconds, or up to 1,000 seconds. The greater the window length, the greater the precision—but also the need for greater system resources. Values of Δt may typically range from 0.001 to 0.1 seconds for ECG applications. Values for n may generally range from about 50 to about 10,000, and base 2 values are common in the art as for example ranges of n from about 64 to about 1,024, or up to 16,384.

The memory locations of values in the magnitude array are referred to as “bins” and represent an incremental frequency bandwidth Δf=1/nΔt. It should be noted that other types of Fourier transforms (FT) are available, such as Discrete Fourier transforms (DFT), but FFT is generally favored for its speed.

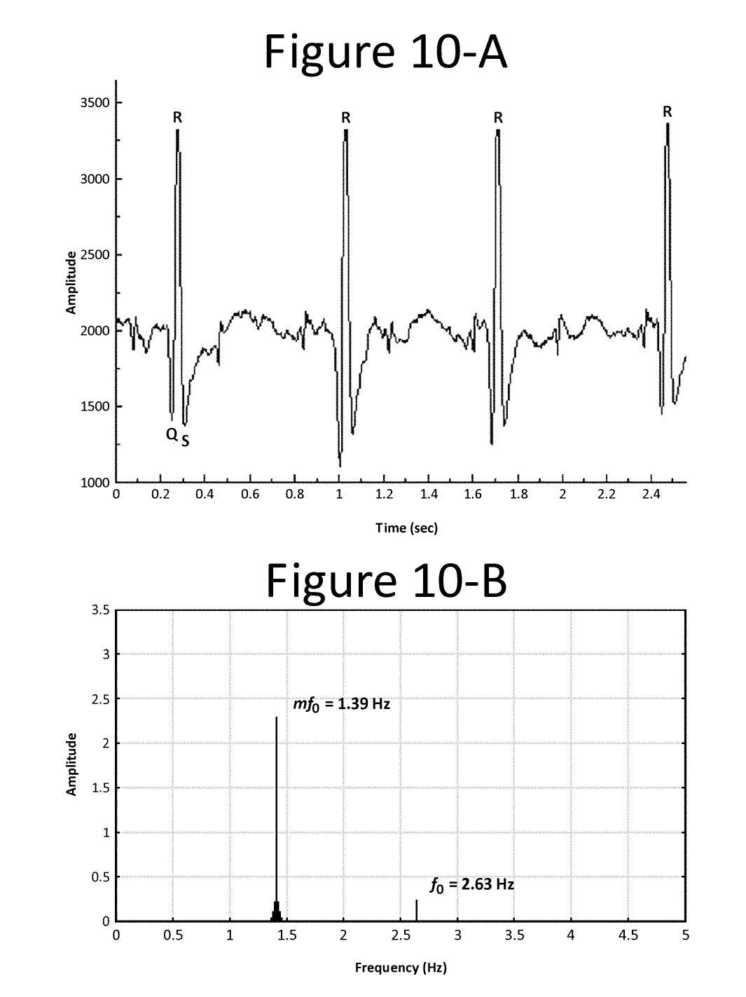

An example of an actual ECG signal is shown in FIG. 10-A. The result of a fast Fourier transform (FFT) is to convert signal data collected by sampling the amplitude at regular time intervals over a selected period of time—a data set in the time domain—into a data representation in the frequency domain (FIG. 10-B), meaning a graph of amplitude versus frequency as opposed to amplitude versus time. In this form, it is a simple matter for the analyzer to go through all the magnitude array elements and locate the maternal and fetal heart rates. Detailed texts on the workings of fast Fourier transforms with code examples may be found in Brigham, E. Oran, The Fast Fourier Transform. Saddle River, N.J.: Prentice Hall, 1973; Smith, Steven W. “Chapter 12 The Fast Fourier Transform.” In The Scientist and Engineer's Guide to Digital Signal Processing, 225-242. Poway, Calif.: California Technical Publishing, 1997; and a discussion of Equation 2 can be found in Longueville, Didier. “Fast Fourier Transform (FFT) (Part 9).” Blog post. Arduinoos. Arduinoos, 8 Mar. 2012. Web. 24 Jan. 2017. <www.arduinoos.com/2012/03/fast-fourier-transform-fft-part-9/—; the disclosures of all of which are incorporated by reference herein in their entirety.

Note, however, that contained in the “raw” ECG signal of FIG. 10-A are not only the maternal and fetal heartbeats, but also noise. Further, the maternal and fetal heartbeats carry their own “noise” in the form of harmonics—Fourier components that oscillate at integer multiples of a fundamental frequency (also known as the 1st harmonic). The maternal PQRST complex comprising the maternal fundamental mf₀ and its higher order Fourier harmonics (plainly revealed by its exceedingly prominent R waves) along with various noise sources, can be seen to effectively swamp the fundamental fetal frequency, f₀, so that it is not at all clear in FIG. 10-A whether or not there even is a fetal heartbeat at all. It should be noted that the ECG of FIG. 10-A is rather unusual in that the Q and S waves of the maternal QRS complex is easily identified. In practice, most maternal P, Q, S, and T waves are often lost in the noise, but the R wave is almost always clearly seen, while the entire fetal PQRST complex is typically obscured.

Referring back to FIG. 8, there is provided at least one variable band-pass filter 845 and at least one variable harmonic notch filter 842, 844 interposed between the ECG signal coming in from the amplifier stage 610 (via multiplexer 620) and a mixed signal input A. This is said to be “mixed signal” because it contains both the fetal fundamental frequency f₀ and the maternal fundamental frequency mf₀. This signal may optionally be tapped off to a variable maternal fundamental (MF) notch filter 840 that strips out the maternal fundamental frequency mf₀ for delivery of the remaining fetal ECG signal, fECG, to fetal signal input B.

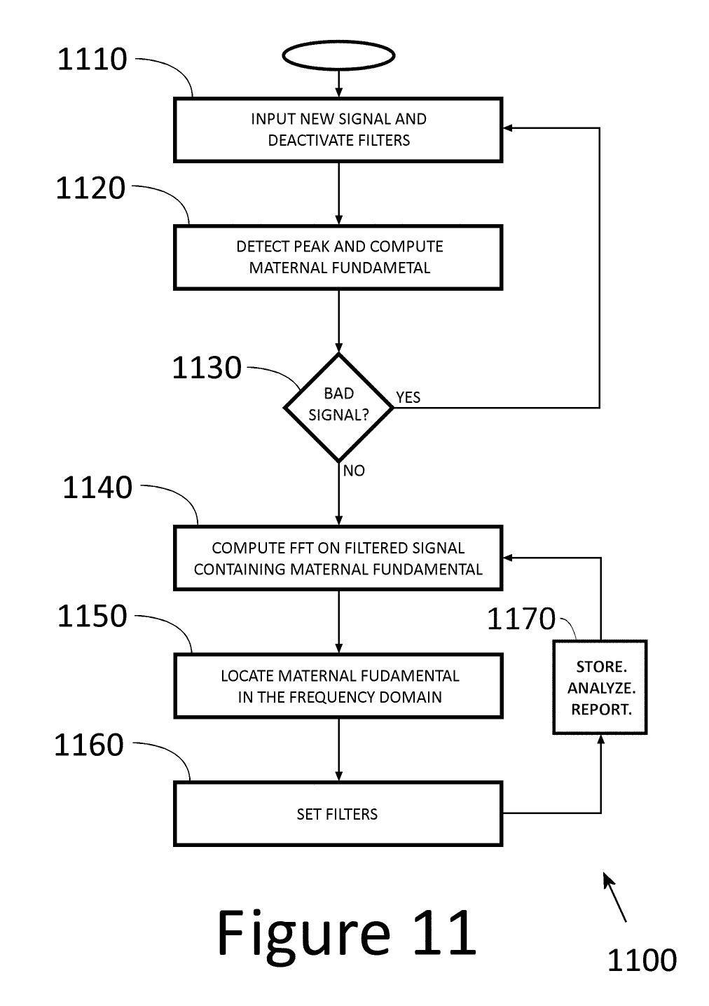

Note, though, that the fECG signal fed into input B is not substantially isolated until all of the maternal harmonics and the maternal fundamental are stripped out, hence the input to the MF notch filter is shown to include mECG*, where the asterisk signifies that this maternal ECG input might range from a total mECG in an initial stage where all the notch filters are inactive to a “refined” mf₀ wherein all the variable harmonic notch filters are precisely tuned. These various stages of filter “tuning” depend on where the system is operating within the flowchart of FIG. 11, to be described below.

When the analyzer 850 has set the filters so as to receive an acceptable fetal heartbeat QRS signal, one use of the signal is to output it to a device 660 that can use the signal to recreate an artificial heartbeat sound that is “live”, meaning the sound is synchronized with the actual fetal heartbeat. The closing of the atrioventricular valves, corresponding to the first of two heartbeat sounds (the “lub” of the “lub-dub” heartbeat sounds) occurs at or near the peak of the R wave. The second heartbeat sound, “dub”, occurs roughly halfway in the decay of the T wave. The T wave in a fetal heartbeat is nearly undetectable, so it is anticipated that the method used would be to execute an artificial lub sound at the peak of an R wave and then to follow with a “dub” after a time delay, where the time delay is inversely related to the measured fetal heartrate. This can be done with software in the analyzer 850 (or analyzers 750 and 650, for that matter), but it may be desired to reduce the load on the analyzer by providing a separate microcontroller for the purpose. Another method is to transmit the fECG signal, or a simplified pulse signal corresponding to the R wave in real time, to a wireless device 670, such as a smart phone, having an app installed to carry out the task.

Hence, the FFT-enhanced analyzer may have two signals available to it for its purposes: a first mixed signal at mixed signal input A comprising both the fetal and maternal fundamentals, and a second fetal signal at fetal signal input B comprising the fetal fundamental, but not the maternal. It should be noted that a signal may not be completely removed by a filter, but instead sufficiently attenuated. Signal filters are rarely perfect. It is enough that a signal be considered “removed” when it has been sufficiently attenuated so as to serve the purposes of the disclosure. A primary purpose of removing frequencies, such as noise and harmonics, is to allow the fetal signal to be revealed and its frequency measured. Among any number of purposes may be to reveal the shape of the fetal waveform where that information is desired, usually for various diagnostic purposes. Yet another may be to store a record of detailed information for archival purposes for whatever future uses a researcher might find of use.

The variable bandpass 845, variable notch 842, 844, and variable maternal fundamental (MF) notch 840 filters are controlled, or “tuned” to their relevant frequencies by the FFT-enhanced analyzer 850 via a plurality of filter control lines 852. Note that the bandpass filter 845 has two relevant frequencies, a low cutoff frequency f_{L} controlled to be set below the maternal fundamental frequency mf₀, and a high cutoff frequency f_{H} controlled to be set above the notch frequency, Nmf₀, of the highest (i.e., the N^{th}, where N is an integer) variable harmonic notch filter 844. The result is that all the ECG frequencies are significantly attenuated except those within the bandpass (BP) bandwidth f_{BP}, namely those above about f_{L} and below about f_{H}. This is also to say that all frequencies at least from the maternal fundamental mf₀ up to the N^{th} maternal harmonic Nmf₀ are passed through as largely or completely unattenuated. The relations are:

f_{L}<mf₀<Nmf₀<f_{H}, for 2mf₀<f_{H} (2)

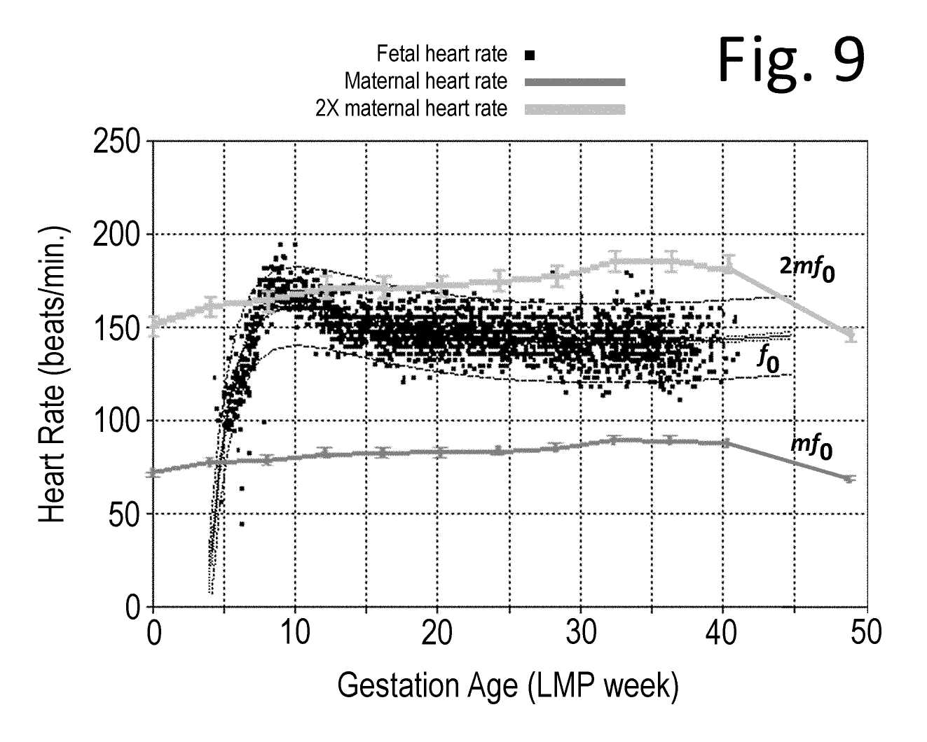

and so it is apparent that all the intervening maternal harmonics 2mf₀, 3mf₀, etc. between mf₀ and Nmf₀ also get through the variable bandpass filter 845. The reason for preferring the high cutoff f_{H} to be greater than the second maternal harmonic 2mf₀ becomes apparent upon examining FIG. 9.

Referring to FIG. 9, there is shown actual experimental results from the literature of fetal and maternal heart rates plotted against time in units of weeks since last menstrual period (LMP). Also plotted is the calculated second maternal harmonic frequency 2mf₀ calculated from the maternal heart rate. The maternal heart rate, of course, is simply the patient's pulse which, when an ECG is taken, is the maternal fundamental frequency mf₀. The second harmonics 2mf₀ are plotted by simply multiplying all the measured maternal pulse rates mf₀ by two. The data for FIG. 9 was derived from and cross-checked with a number of sources, specifically DuBose et al. Sonographic Correlation of Fetal Heart Rate and Gender. J. Diag. Med. Sonography, 1989; 5(2):49-53; DuBose et al. Embryonic Heart Rate and Age, JDMS, 1990; 6(3):151-157; DuBose et al. JUM, 7:237-238, 1988; DuBose, Terry J. “Chapter 12 Heart Rate.” Fetal Sonography. 1st ed. Philadelphia: W. B. Saunders, 1996. 263-74. Print; Hunter and Robson. Adaption of the maternal heart in pregnancy. Br. Heart J. 1992; 68; and Atkins, A. F. J. et al. “A Longitudinal Study of Cardiovascular Dynamic Changes throughout Pregnancy.” Europ. J. Obstet. Gynec. Reprod. Biol. 12 (1981): 215-24. Print.

As can be seen, the vast majority of fetal heartbeats f₀ plotted fall between the 1^{st} and 2^{nd} maternal harmonics. In order to ensure that at least the 1^{st} fetal harmonic f₀ (the fetal fundamental) makes it through the bandpass filter 845 it would be desirable to set f_{H} substantially higher than mf₀, such as perhaps around 2.5mf₀ or higher, depending on how steep the cutoff curve is for the bandpass filter 845 design.

Referring to FIG. 11, there is shown a flowchart of an embodiment of a FFT tracking method 1100 of the disclosure which may be executed using fetal heart monitors of the type shown in FIG. 8. Here, the flowchart picks up at node 1110 where a new signal is obtained, such as one newly selected by the channel scanner/selector 625 and fed in through the multiplexer 620. The variable filters 840, 842, 844, 845 are all deactivated here, which may mean that they are shut off completely or set to frequencies and bands well out of any ranges of interest. Hence, a raw ECG signal such as exemplified in FIG. 10-A (or top graph of FIG. 5) is fed through unimpeded and unadulterated to input A of the FFT-enhanced analyzer 850 (and optional MF notch filter 840).

At node 1120 the FFT-enhanced analyzer 850 may use FFT, classical, or both means to make an initial determination of what the maternal fundamental frequency mf₀—the mother's pulse rate—is. The FFT means is to examine the output of the FFT module 855 and find the frequency bin containing the greatest computed magnitude. A classical means may be to use any one of the number of “peak detectors” well known in the classic electronic art to isolate the signal with the greatest magnitude, namely the maternal R wave, and determine its frequency. Another classical means is an analog to digital conversion of the signal followed by a numeric scan to find the peaks. Applying both an FFT and a classical method may be useful as a verification procedure.

It may be that the signal is bad and no maternal R signal can be detected, which is not unusual in the ECG art. In such a case, at node 1130 control flows back to node 1110 where the FFT-enhanced analyzer 850 commands the channel scanner/selector 625 to pipe in another signal.

When a good signal is found with a detectable maternal R wave, control eventually flows to node 1140 where a fresh FFT is performed and the maternal fundamental mf₀ is determined at node 1160.

At node 1160, the value of the maternal fundamental mf₀ is used to calculate the signals required to set the notch frequencies mf₀, 2mf₀, 3mf₀, . . . Nmf₀ of the variable notch filters 840, 842, 844 and the low and high cutoff frequencies f_{L} and f_{H} of the variable bandpass filters 845. Control is then returned to 1140 thereby providing a continuous loop of constant FFT monitoring of the ECG signal, constant tracking of the maternal heart rate, and constant readjustment of the variable filters 840, 842, 844, 845. This infinite loop may be periodically exited by conventional interrupt means as needed or desired.

So long as the “infinite” loop of FIG. 11 operates, a continuous monitoring of the fetal heart rate is provided at node 1170, where the results are stored, analyzed, and reported the output/receiving devices 660, 670. It is anticipated that the analyzer may be programmed to detect an abnormal heartbeat and issue an alarm signal. Alternatively, or as a backup, any of the output/receiving devices may be programmed or wired to detect an abnormality in the fetal heartbeat and issue an alarm signal.

In operation, an FFT output such as shown in FIG. 10-B should be obtained from the Fourier transform on the signal at mixed signal input A, though it should be known that FIG. 10-B is idealized. There will be some noise, but the fetal fundamental f₀ should be apparent. The signal is mixed in that the maternal fundamental is present, but optionally a pure fetal signal fECG may be generated by tapping off the mixed signal at input A and running it through the maternal fundamental variable notch filter 840, thereby stripping out the maternal signal mECG and delivering the fetal signal fECG to input B for use by the FFT-enhanced analyzer 850 as desired or needed.

One of the results of the FFT system disclosed herein is that filtering of the ECG signal is accomplished by hardware filtering instead of software filtering. Software filtering would have required that (1) noise and maternal harmonics in the FFT results be identified, (2) the magnitudes of the noise and maternal harmonics deleted, and (3) an inverse FFT executed to reconstruct a filtered ECG signal in the time domain. To complete such a process would require that an array of phase angles as well as magnitudes be computed. This requires extensive CPU power and memory and much time, which also implies a larger and bulkier monitor controller 150. Many microcontrollers, such as the Atmel® ATmega328 famously utilized in the Arduino Uno, are incapable of computing arctangents and therefore incapable of computing FFT phase angles at all. One could, of course, provide an FFT coprocessor, field programmable gate array (FPGA) or the like, but the hardware costs, space, and power requirements increase substantially.

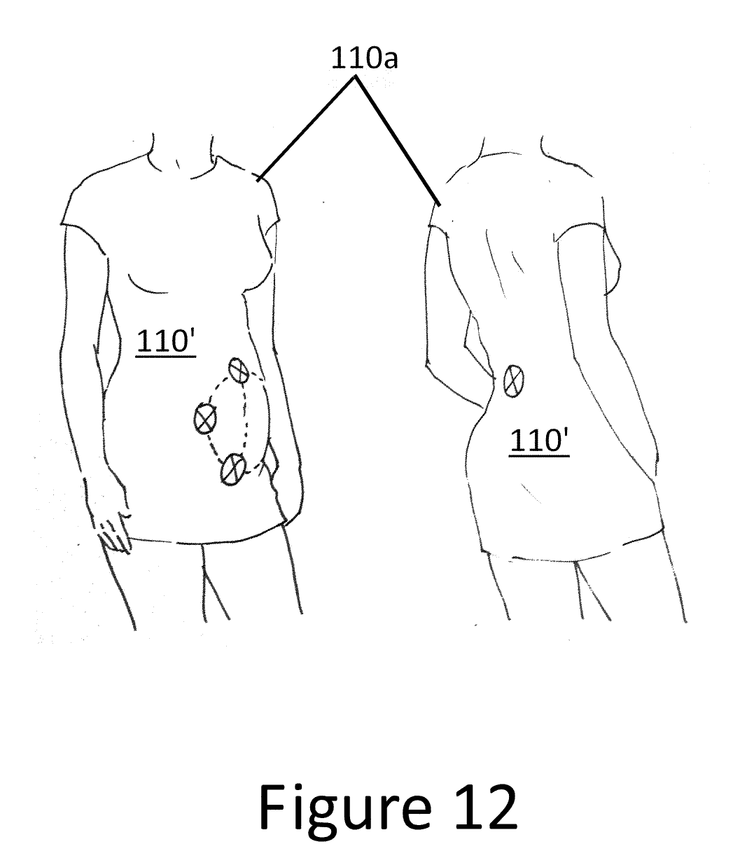

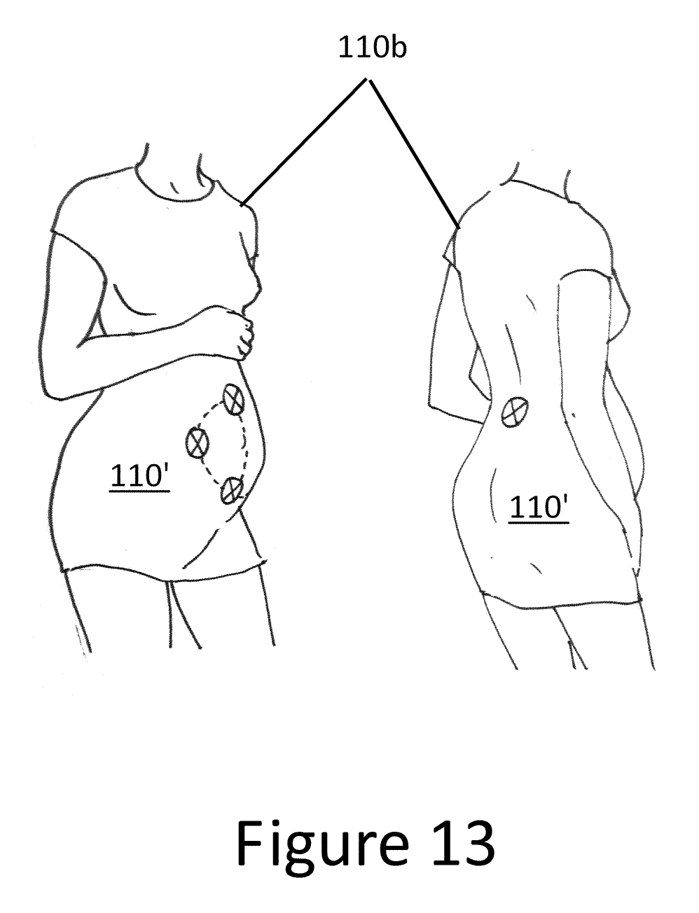

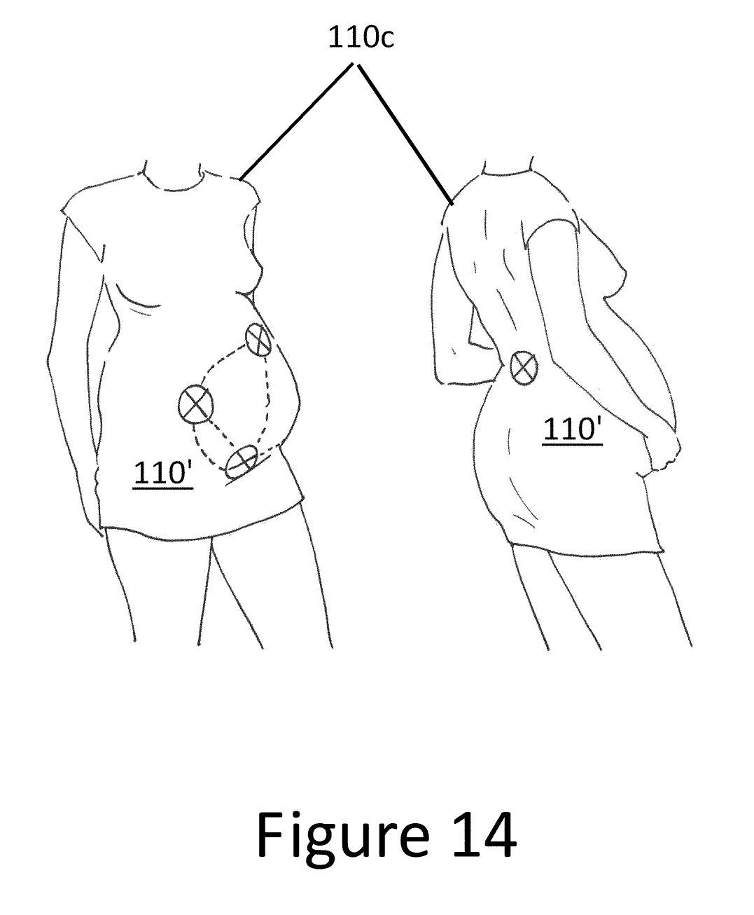

Referring to FIGS. 12 through 13, one may provide the embodiments of the disclosure available in a pregnancy kit form, wherein the garment 110 is made available in a plurality of waist sizes 110a, 110b, 110c, . . . and so forth to track the increasing size of the expectant mother's abdomen as the pregnancy advances. This would be used to avoid the abdomen size from exceeding the stretch capabilities of the fabric 110′ that the garment is constructed of. What sizes would be made available and how many has some dependency then on the fabric 110′ selected. The objective is to ensure good contact between the expectant mother and the electrodes 120 throughout as much of the monitoring period as practicable.

The pregnancy kit would include a least one monitor controller 150 that would simply be unplugged from an outgrown garment 110 (e.g., 110b) and plugged in to the next larger size garment 110 (e.g., 110c).

The foregoing disclosures relate to illustrative embodiments of the invention and modifications may be made without departing from the spirit and scope of the invention as set forth in, and limited only by, the claims herein.

In the claims herein—unless explicitly indicated otherwise—the use of the word “or” is to be construed as the inclusive “or” in accordance with common usage in the engineering art.

What is claimed is:

1. A method of measuring a fetal heartbeat, comprising the steps of:
continuously inputting an ECG signal from a female;
continuously performing a Fourier analysis on said ECG signal effective in locating a fundamental maternal frequency of the heartbeat of the female in a frequency domain derived thereby;
continuously adjusting at least one variable filter so as to substantially attenuate a harmonic of said determined fundamental maternal frequency; and
continuously locating the fetal heartrate thus revealed in said frequency domain.

2. A fetal heart monitor, comprising:
an FT-enhanced analyzer having a Fourier transform capability;
one or more variable harmonic notch filters, controllable by said FT-enhanced analyzer; and
said one or more variable harmonic notch filters interposed between and in communication with a first signal input to said FT-enhanced analyzer and a signal-source derived from at least one ECG electrode.

3. The apparatus of claim 2, further comprising:
a multiplexer controllable by said FT-enhanced analyzer;
wherein said multiplexer receives as input the signals from two or more of said ECG electrodes and outputs the signal of a currently selected ECG electrode to said one or more variable harmonic notch filters.

4. The apparatus of claim 2 wherein said Fourier transform capability comprises a Fast Fourier Transform capability.

5. The apparatus of claim 4 wherein said Fast Fourier Transform capability comprises a Fast Fourier Transform co-processer.

6. The apparatus of claim 2 further comprising a variable maternal fundamental notch filter interposed between and in communication with a second signal input to said FT-enhanced analyzer and said signal-source derived from at least one ECG electrode.

7. The apparatus of claim 6 further comprising means for creating an audible artificial fetal heartbeat from the signal received at said second signal input.

8. The apparatus of claim 2 wherein said FT-enhanced analyzer executes the method of claim 1.

9. A fetal heart monitor vestment, comprising:
a non-conductive fabric garment configured to be closely fitted to a female;
a monitor controller unit comprising a fetal heart rate monitor and having a data input port;
an ECG harness comprising a plurality of electrodes and a plurality of conductive fabric wires, each electrode attached to at least one of the conductive fabric wires;
the conductive fabric wires in attachment to and in communication with the data input port;
the electrodes and conductive fabric wires integrated into the fabric of the garment; and
at least one said electrode disposed in the garment so as to be positioned on, and in close contact with, the female in a manner effective in sensing a fetal cardiac potential signal.

10. The apparatus of claim 9 wherein said electrodes are of a conductive fabric.

11. The apparatus of claim 9 wherein said electrodes are embroidered electrodes of a conductive thread.

12. The apparatus of claim 9 wherein the non-conductive fabric of said garment and the conductive fabric of said electrodes demonstrate substantial similar stretch characteristics.

13. The apparatus of claim 9 in a pregnancy kit form comprising two or more waist sizes of said fetal heart vestment.

14. The apparatus of claim 9 wherein said fetal heart rate monitor executes the method of claim 1.
